(19) United States
(12) Patent Application Publication
(10) Pub. No.: US 2024/0423577 A1
(43) Pub. Date: Dec. 26, 2024

(54) DYNAMIC TOMOSYNTHESIS SYSTEM AND VENTILATION AND PERFUSION IMAGING SYSTEMS AND METHODS EMPLOYING SAME

(71) Applicant: KONINKLIJKE PHILIPS N.V., EINDHOVEN (NL)

(72) Inventors: Thomas Koehler, Hamburg (DE); Roberto Buizza, Eindhoven (NL); Rafael Wiemker, Hamburg (DE); Cornelis Petrus Hendriks, Eindhoven (NL); Joerg Sabczynski, Hamburg (DE); Jaap Roger Haartsen, Best (NL); Nataly Wieberneit, Hamburg (DE); Michael Polkey, London (GB)

(21) Appl. No.: 18/605,894

(22) Filed: Mar. 15, 2024

Related U.S. Application Data
(60) Provisional application No. 63/522,734, filed on Jun. 23, 2023.

(51) Int. Cl. A61B 6/00; A61B 6/02; A61B 6/40; A61B 6/50

(52) U.S. Cl. CPC A61B 6/54; A61B 6/025; A61B 6/4007; A61B 6/4435; A61B 6/507; A61B 6/5205; A61B 6/5217

(57) ABSTRACT

X-ray sources emit X-rays into an examination region along different projection views. An X-ray detector array detects the X-rays emitted by the X-ray sources after passing through the examination region. X-ray imaging data are acquired by cycling through the X-ray sources with each step of the cycle including: switching an active X ray source on to emit X-rays and the other X ray sources off to not emit X rays, and acquiring X ray imaging data along the projection view corresponding to the active X-ray source that is switched on. Tomosynthesis image reconstruction is performed on the X ray imaging data to generate at least one volumetric image. A time sequence of spatially aligned images is produced from the time sequence of volumetric images. A perfusion image and/or a ventilation image is generated based on voxel intensity variation over time of the time sequence of spatially aligned images.

### CROSS-REFERENCE TO RELATED APPLICATIONS

This patent application claims the priority benefit under 35 U.S.C. § 119 (e) of U.S. Provisional Application No. 63/522,734, filed on Jun. 23, 2023, the contents of which are herein incorporated by reference.

The following relates generally to the medical imaging arts, pulmonary imaging arts, medical image processing arts, pulmonary image processing arts, medical imaging driven medical diagnostic and treatment guidance arts, medical imaging driven pulmonary diagnostic and mechanical ventilation therapy arts, and related arts.

### BACKGROUND

A quantitative and spatially resolved assessment of the lung's ventilation and perfusion is desired to optimize the ventilator settings and other therapy parameters in mechanically ventilated patients. Usually, the aim is to match ventilation and perfusion (aka. V-Q matching) as ventilation and perfusion are equally important for proper gas exchange in the alveoli.

Ventilation and perfusion imaging can be done using computed tomography (CT), positron emission tomography (PET), magnetic resonance imaging (MRI), electrical impedance tomography (EIT) or scintigraphy. However, these techniques are not well suited to be used in the intensive care unit (ICU) due to workflow issues. For example, CT, PET, and MRI systems are typically stationary, so that the patient needs to be moved to the radiology department. Scintigraphy requires a radioactive contrast agent, which requires careful handling and specially trained operators. Although EIT can be used in the ICU, it has a much lower resolution than the other modalities mentioned.

Recently, the technique of dynamic X-ray imaging has emerged to address this task. As the lung inflates during inhalation, the average density of lung tissue decreases. By tracking the attenuation of image regions during the respiratory cycle, the local ventilation can be estimated. This technique is sometimes referred to as ventilation imaging and can provide spatially resolved information on air flow within the lungs. In a similar manner, perfusion imaging can be performed to provide spatially resolved information on pulmonary blood flow in the lungs. In the systolic phase of the cardiac cycle, blood is flowing into the lung tissue, leading to an increased density of the lung tissue, while during the diastolic phase blood flows out of the lung tissue leading to decreased density. By tracking the attenuation during the cardiac cycle, the local perfusion can be estimated. However, dynamic X-ray imaging is sensitive to patient motion causing anatomical features to overlap differently during the respiratory cycle and can be difficult to implement with sufficient imaging accuracy in a bedside setting. Computed tomography (CT) imaging can provide dynamic Xray imaging with improved imaging quality but is typically not feasible as a bedside imaging technique. Particularly in the case of mechanically ventilated patients (who are prime candidates to benefit from ventilation and perfusion imaging), transport of the mechanically ventilated patient to and from the radiology department is cumbersome.

The following discloses certain improvements to overcome these problems and others.

### SUMMARY

In one aspect, a medical imaging system includes: X-ray sources arranged to emit X-rays into an examination region along different respective projection views spanning less than 180 degrees; an X-ray detector array arranged to detect the X-rays emitted by the X-ray sources after passing through the examination region; and an electronic controller configured to perform an imaging method. The imaging method may in some embodiments include: acquiring X-ray imaging data by cycling through the X-ray sources; and performing tomosynthesis image reconstruction of the X ray imaging data to generate at least one image. Each step of the cycle including: switching an active X ray source on to emit X-rays and the other X ray sources off to not emit X rays, and acquiring X ray imaging data along the projection view corresponding to the active X-ray source that is switched on.

In another aspect, a medical imaging method is performed using X-ray sources arranged to emit X-rays into an examination region along different respective projection views spanning less than 180 degrees, and an X-ray detector array arranged to detect the X-rays emitted by the X-ray sources after passing through the examination region. The medical imaging method includes cycling through the X-ray sources, with each step of the cycle including: switching an active X ray source on to emit X-rays and the other X ray sources off to not emit X rays, and acquiring X ray imaging data along the projection view corresponding to the active X-ray source that is switched on. The medical imaging method further includes performing tomosynthesis image reconstruction of the acquired X ray imaging data to generate at least one image.

In either of the above aspects, the tomosynthesis image reconstruction may include grouping the X-ray imaging data into acquisition time intervals, and performing tomosynthesis image reconstruction of the X-ray imaging data grouped into each time interval to generate a time sequence of images. In some further aspects, the time sequence of images may depict at least one in vivo lung, and the method may further include and generating a perfusion image and/or a ventilation image based on voxel intensity variation over time of the time sequence of images.

One advantage resides in providing improved dynamic X-ray imaging (e.g., perfusion and/or ventilation imaging) of a patient.

Another advantage resides in providing for bedside dynamic X-ray imaging with improved image quality.

Another advantage resides in providing four-dimensional (three-dimensional spatial plus time) dynamic Xray imaging with temporal resolution sufficient to perform perfusion imaging, without the use of a complex and typically stationary CT scanner.

Another advantage resides in providing a tomosynthesis Xray imaging system with high temporal resolution, for example sufficient to perform perfusion imaging of the lungs.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an imaging system in accordance with the present disclosure.

FIGS. 2 and 3 diagrammatically show flowcharts of an imaging method using the imaging system of FIG. 1.

FIGS. 4-6 show examples of processing operations to generate 4D data by the system of FIG. 1.

### DETAILED DESCRIPTION

As used herein, the singular form of “a,” “an,” and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

Tomosynthesis X-ray imaging can provide three-dimensional (3D) spatial images (albeit with coarse resolution along the general direction of the X-ray beams, hence sometimes referred to as 2.5D spatial image), and optionally four-dimensional (4D) imaging (3D plus time) without the complexity of a CT scanner. The general concept of tomosynthesis is to acquire X-ray images over a limited angular range, i.e., less than 180°, providing at least coarse depth resolution in the direction running between the X-ray source and the X-ray detector. Tomosynthesis Xray imaging is used, for example, in the field of X-ray mammography. Tomosynthesis imaging is distinguished from tomographic imaging, such as that performed using a CT scanner, in that tomographic imaging requires X-ray imaging data be acquired over at least 180°, and typically a CT scanner will provide imaging data over a full 360° using a rapidly rotating gantry (e.g., 120 revolutions per minute in some commercial CT scanners). CT imaging can thus provide 4D imaging at high resolution. However, a CT scanner is a complex electromechanical system with a high speed rotating gantry, and is generally not available as a mobile bedside unit.

In tomosynthesis imaging, on the other hand, the X-ray source only traverses a smaller angular range (less than 180° and more commonly an angular range of 15°60°). This reduces mechanical complexity and overall size of the imaging system, and facilitates use of tomosynthesis imaging in applications such as mammography. However, because the X-ray source does not perform full 360° revolutions, the gantry for tomosynthesis imaging moves the Xray source bidirectionally, e.g., in a +angular direction and then back in an angular direction. This means the X-ray gantry for tomosynthesis cannot be a high-speed rotating gantry that performs rapid full 360° revolutions. This, in turn, limits temporal resolution of the tomosynthesis X-ray system. For example, a typical tomosynthesis X-ray imaging system cannot acquire tomosynthesis X-ray imaging data with sub-second (or more preferably millisecond) temporal resolution to enable imaging over the course of a cardiac cycle so as to perform perfusion imaging of the lungs, or even the slightly coarser temporal resolution (determined by the time for a single breath) for pulmonary imaging.

There are, furthermore, several shortcomings and limitations of dynamic x-ray imaging for ventilation and perfusion imaging. One of the main challenges is that the changes in attenuation in a certain image pixel on the detector caused by ventilation and perfusion are typically small and they are superimposed by motion. In particular, motion of high-contrast structures like the diaphragm, the ribs, the heart wall, and the vessels in the lung can easily cause a much stronger change of the attenuation seen by a particular detector pixel. Thus, sophisticated image processing is needed to estimate ventilation and perfusion: ribs are removed by dedicated image processing followed by elastic image registration to compensate at least for the main motion of the lung tissue. However, the lung is a very soft and flexible organ, and it moves on a static background (dorsal part of the thorax) and its motion is also overlayed by the more slowly moving chest wall.

Another limitation of dynamic x-ray imaging is that it provides only 2D projective information, while in the use case of mechanically ventilated patients, it is also desired to visualize the ventilation in the ventral-dorsal direction.

The following discloses a medical imaging system that combines the technologies of dynamic x-ray imaging and tomosynthesis. The above limitations on tomosynthesis imaging could be remediated by use of tomographic X-ray imaging, but as just noted tomographic X-ray imaging typically does not provide sufficient temporal resolution for perfusion lung imaging or even ventilation imaging. Embodiments disclosed herein provide tomosynthesis X-ray imaging using electronic cycling between multiple Xray sources to provide tomosynthesis X-ray imaging with temporal resolution sufficient for dynamic X-ray imaging of the lungs. This results in dynamic images showing the density variations of dynamic x-ray imaging with the spatial characteristics of tomosynthesis images (i.e., 2D “slices” instead of projections), and sufficiently high temporal resolution to resolve density changes in time due to ventilation and perfusion of the lungs. Two nonlimiting illustrative embodiments include a linear or a circular tomosynthesis acquisition, respectively. In order to reach the required temporal resolution on the order of 100 milliseconds (ms) for perfusion imaging, the X-ray detector should be fast to provide X-ray images at a rate of at least 100 Hz, which would provide 10 images within the temporal window of 100 ms.

To achieve this, in some embodiments CMOS-based detectors (instead of a CCD-based onc) or a photon-counting approach (e.g., using a perovskite (see, e.g., L. Pan, “Perovskite CsPbBr3 single crystal detector for high flux X-ray photon counting and synchrotron X-ray detection”, Proceedings of the SPIE, 2022, https://doi.org/10.1117/12.2633070 as direct conversion material) is used to provide the desired framerate. The detector optionally has a low-resolution (compared to the standard resolution of detectors used in chest x-ray) to reduce data bandwidth requirements. The disclosed high speed tomosynthesis imaging systems further use a set of small x-ray tubes with so-called cold emitters like carbon nanotubes (see, e.g., J. S. Han et al., “High-Performance Cold Cathode X-ray Tubes Using a Carbon Nanotube Field Electron Emitter,” ACS Nano 2022; 16(7): 10231-10241, https://doi.org/10.1021/acsnano.2c02233), which can be turned on and off within less than a millisecond in some embodiments.

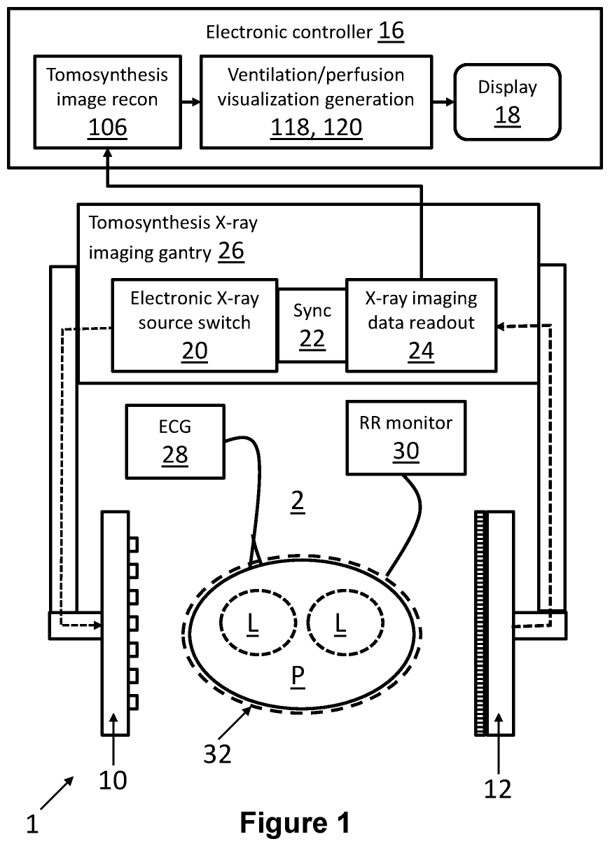

With reference to FIG. 1, a nonlimiting illustrative example of such a medical imaging system 1 is diagrammatically shown. The medical imaging system 1 is an X-ray imaging device. As shown in FIG. 1, the X-ray imaging system 1 includes an array of X-ray sources 10 (e.g., Xray source 10₁, 10₂, . . . , 10_{N1}, 10_{N} as indicated in FIG. 1, where N is the number of X-ray sources in the array 10) arranged to emit X-rays into an examination region 2 of the medical imaging system 1 where a patient P is disposed for an imaging examination along different respective projection views spanning less than 180 degrees (e.g., less than 90 degrees). As shown in FIG. 1, the X-ray sources 10 are arranged as a linear array of N X-ray sources 10₁, 10₂ . . . , 10_{N1}, 10_{N}, but more generally can have any suitable arrangement, such as N X-ray sources arranged as a loop. The Xray sources 10 are configured to be electronically switched on to emit X-rays and electronically switched off to not emit X-rays. To do so, the X-ray sources 10 have a switching speed of 10 milliseconds or faster. For example, the X-ray sources 10 can comprise cold-cathode X-ray tubes, and in particular can include carbon nanotube (CNT) field emitters.

The medical imaging system 1 also includes an X-ray detector array 12 arranged to detect the X-rays emitted by the X-ray sources 10 after passing through the examination region 2. In some embodiments, the X-ray detector array 12 comprises complementary metal oxide semiconductor (CMOS)-based detectors or direct conversion X-ray detectors, which as previously mentioned provide desirably high speed, e.g., to provide and X-ray image acquisition rate of 100 Hz or faster in some embodiments. During operation to acquire imaging data of the patient P or other imaging subject, that imaging subject P is disposed in the examination region 2. The illustrative medical imaging system 1 employs tomosynthesis imaging in which the X-ray sources 10 are cycled on and off as disclosed herein. In some configurations, the array of X-ray sources 10 and detector 12 can be moved on robotic arms or the like to different vantage points (called “views”) around the patient to (for example) provide clinically significant views of lungs, or in an image guided therapy (IGT) system to provide a chosen view of an interventional procedure. However, as previously discussed, such robotic/mechanical rotation of the X-ray sources is typically insufficiently fast to provide a frame rate sufficient for lung perfusion imaging or ventilation imaging. Rather, switching between the Xray sources 10₁, 10₂ . . . , 10_{N1}, 10_{N} provides the effective angular “rotation” for 4D tomosynthesis imaging with sub-second (e.g., millisecond) frame rates suitable for lung perfusion imaging or ventilation imaging.

In illustrative FIG. 1, the X-ray sources 10 have a planar arrangement, and similarly the X-ray detector array 12 is a planar array. However, it is contemplated to arrange the X-ray sources 10 on a curved surface to provide for the X-ray sources to partially arc around the patient P, and similarly the X-ray detector array 12 could be a curved array to partially arc around the patient P in a complementary fashion.

In the illustrative example of FIG. 1, the X-ray imaging system 1 further includes an overhead gantry 14 having a first descending arm 141 on which is mounted the array of X-ray sources 10 and a second descending arm 142 on which is mounted the X-ray detector array 12, with the array of X-ray sources 10 and the X-ray detector array 12 both facing toward the examination region 2 so that X-rays from the Xray sources 10 impinge on the X-ray detector array 12 after passing though the examination region 2. The first and second descending arms 141 and 142 (and hence the attached respective X-ray sources 10 and detector array 12) are spaced apart to accommodate a human subject (e.g., patient P) disposed in the examination region 2 in a prone or supine position between the X-ray sources 10 and the X-ray detector 12. The illustrative X-ray imaging system 1 can thus advantageously be utilized to image the patient P lying on a (diagrammatically indicated) bed 15 in a supine or prone position.

The tomosynthesis Xray imaging system 1 in some embodiments constitutes a bedside imaging system 1 (for example, with the gantry 14 mounted on wheels, not shown) that can simultaneously image both lungs L (that is, both the left lung L_{L} and the right lung L_{R}) of the patient P. This can be done without positioning a flat panel X-ray detector underneath the patient P (requiring lifting the patient P) or below the bed 15 (where the bed can absorb X-rays and degrade the X-ray image quality). Without tomosynthesis imaging, an X-ray image acquired with this geometry would result in a single two-dimensional (2D) image that would effectively present a superposition of the left and right lungs. However, the tomosynthesis image reconstruction implemented via cycling of the multiple Xray sources 10₁, 10₂ . . . , 10_{N1}, 10_{N} as disclosed herein provides at least coarse spatial resolution along the lateral direction of the supine or prone patient P (or, more generally, along the general direction extending between the X-ray sources 10 and detector 12). The tomosynthesis imaging thus can provide simultaneous acquisition of images of both the left and right lungs L_{L} and L_{R} with the lateral arrangement shown in FIG. 1. The tomosynthesis X-ray imaging provides improved spatial resolution, along with high temporal resolution tomosynthesis imaging provided by electronic switching between the multiple Xray sources 10₁, 10₂ . . . , 10_{N1}, 10_{N} to enable implementation of advanced clinical Xray lung imaging techniques such as ventilation and perfusion imaging of the lungs.

While comparable imaging capability can be obtained using a computed tomography (CT) scanner, such a CT scanner is mechanically complex and typically is not a mobile imaging device. Hence, the patient undergoing a CT scan is transported from the hospital room to the radiology laboratory via a gurney, and then back to the hospital room. Especially in the case of a mechanically ventilated patient, such transport is a complex process performed by a team of clinicians and may significantly stress the patient. By contrast, the illustrative mobile tomosynthesis Xray imaging system 1 of FIG. 1 with its overhead gantry 14 can in some embodiments provide simultaneous bedside imaging in the hospital room of the patient of both left and right lungs L_{L} and L_{R}, thus facilitating use of tomosynthesis X-ray imaging system 1 in providing daily (or otherwise frequent) assessment of the condition of the lungs L of the patient and the mechanical ventilation efficacy.

While the illustrative tomosynthesis X-ray system 1 is designed to provide bedside imaging as discussed above, tomosynthesis imaging systems as disclosed herein can be implemented in other configurations (not illustrated). In one alternative configuration, the array of Xray sources 10 is mounted overhead, that is, above the patient, for example on an L-shaped arm extending over the patient. The overhead X-ray sources emit X-rays generally downward, and the X-ray detector 12 is suitably a flat panel detector that is placed underneath the patient P or underneath the bed 15, facing upward to detect the downwardly directed X-rays after they pass through the patient.

As another nonlimiting example, the tomosynthesis X-ray system could be implemented as a C-arm-imaging system, that is, with the array of X-ray sources 10 disposed on one end of the C-arm- and the X-ray detector array 12 disposed on the opposite end. In such a C-arm configuration, the C-arm can be movable to provide different views of the patient, thus allowing tomosynthesis imaging to be performed at multiple views provided by movement of the C-arm. However, the C-arm movement is typically too slow to provide tomosynthesis image acquisition with sub-second or millisecond temporal resolution. Rather, at each view set via the C-arm-, the multiple X-ray sources 10₁, 10₂ . . . , 10_{N-1}, 10_{N} are cycled to provide the tomosynthesis image acquisition with a subsecond- or millisecond frame rate.

With continuing reference to FIG. 1, the X-ray detector array 12 is in electronic communication with an electronic device controller 16, such as a workstation computer, a server computer, or more generally a computer. Images produced by the medical imaging system 1 via the X-ray radiation generated by the X-ray sources 10 are processed by the device controller 16. For example, in a common configuration the device controller 16 is provided for controlling the imaging device 1 to perform image acquisition. The device controller 14 includes an electronic processor (e.g., a microprocessor), and may in some embodiments include at least one user input device (e.g., a mouse, a keyboard, a trackball, and/or the like-all of which are not shown) for user control of the X-ray imaging system 1. A display device 18 (e.g., an LCD display, plasma display, cathode ray tube display, and/or so forth) provides for displaying Xray images acquired by the X-ray imaging system 1, and/or for displaying processed images therefrom such as lung perfusion and/or ventilation images.

The disclosed imaging system 1 is further configured as described above to perform an imaging method or process 100. In some examples, the method 100 may be performed at least in part by cloud processing. The imaging method or process 100 may, for example, be used to acquire lung perfusion and/or ventilation images.

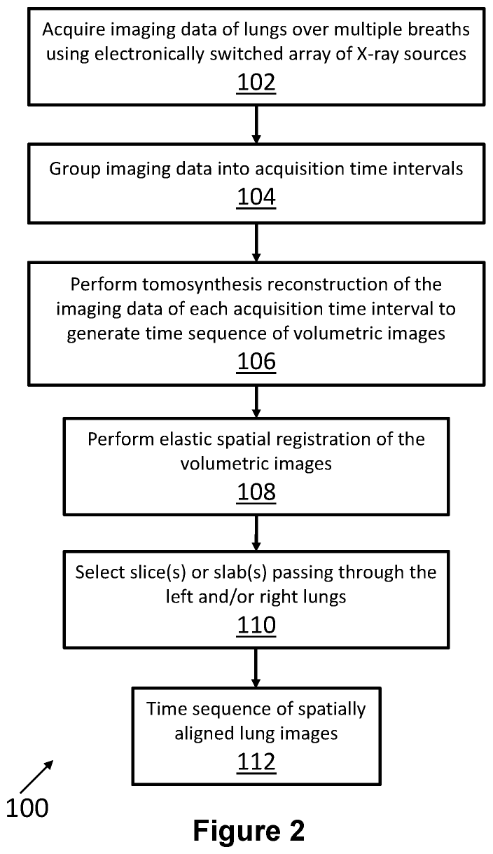

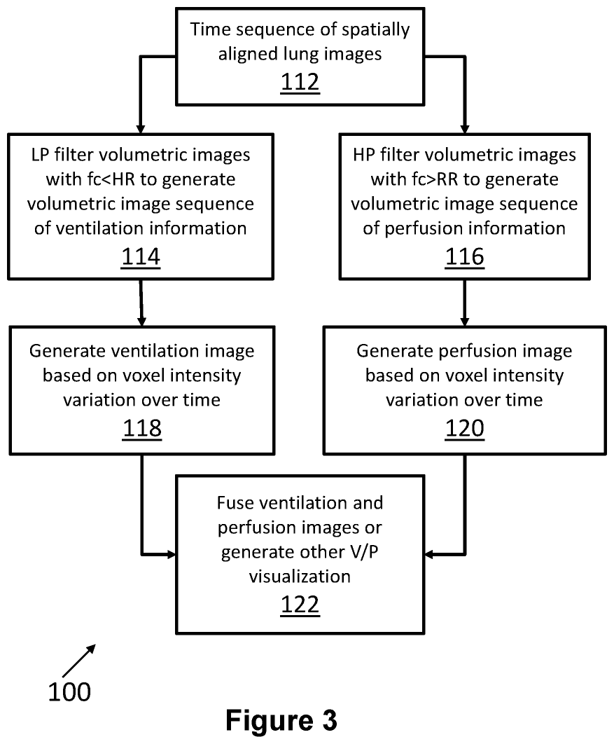

With reference to FIGS. 2 and 3, and with continuing reference to FIG. 1, an illustrative embodiment of an instance of the imaging method 100 is diagrammatically shown as a flowchart. To begin the method 100, the patient P is positioned in the examination region 2 of the medical system 1. At an operation 102, the electronic controller 16 is configured to control the X-ray sources 10 and the X-ray detectors 12 to acquire X-ray imaging data of lungs L over multiple breaths using the electronically switched array of X-ray sources 10.

To acquire the tomosynthesis imaging data, the electronic controller 16 is configured to control an electronic X-ray source switch 20 to cycle through the X-ray sources 10. Each step of the cycle includes switching an active Xray source 10 on to emit X-rays and the other Xray sources 10 off to not emit X-rays. For example, in the first step of the cycle, the X-ray source 10₁ may be switched on to emit X-rays and the other X-ray sources 10₂, . . . , 10_{N1}, and 10_{N} may be switched off. Thus, only the first Xray source 10₁ is used, and the Xray imaging data is acquired along the projection view corresponding to the active X-ray source 10₁ that is switched on. In the next step of the cycle, the X-ray source 10₂ may be switched on to emit X-rays and the other X-ray sources 10₁, 10₃, . . . , 10_{N1}, and 10_{N} may be switched off. Thus, in the second step only the second Xray source 10₂ is used, and the Xray imaging data is acquired along the projection view corresponding to the now-active X-ray source 10₂. This projection view is slightly shifted versus the projection view of the first step of the cycle, due to the physically different position of the X-ray source 10₂ compared with the position of the X-ray source 10₁. This cycling continues through the X-ray sources of the array 10. In the next-to-last step of the cycle, only the second-to-last Xray source 10_{N1} is on and the other X-ray sources 10₁, 10₂, . . . 10_{N2}, and 10_{N} are switched off, and the Xray imaging data is acquired along the projection view corresponding to the now-active X-ray source 10_{N1}. Finally, in the last step of the cycle, only the last Xray source 10_{N} is on and the other X-ray sources 10₁, 10₂, . . . , and 10_{N1} are switched off, and the Xray imaging data is acquired along the projection view corresponding to the now-active X-ray source 10_{N}. As the X-ray sources 10 have subsecond, and preferably millisecond (e.g. 10 milliseconds or faster) switching speed, and the X-ray detector array 12 also has high-speed (e.g. CMOS-based or direct conversion) detectors providing X-ray image frame rates on the order of 100 Hz or faster, the described cycle through the N X-ray sources 10₁, 10₂, . . . , 10_{N1}, 10_{N} can be performed rapidly, e.g. in a fraction of a second, and this cycle can then be repeated, that is, successively using X-ray sources 10₁, 10₂, . . . , 10_{N1}, 10_{N}, 10₁, 10₂, . . . , 10_{N1}, 10_{N}, 10₁, 10₂, . . . , 10_{N1}, 10_{N}, . . . in order to acquire tomosynthesis X-ray imaging data with sub-second frame rates and spanning an angular range provided by the physical distance covered by the N X-ray sources 10₁, 10₂, . . . , 10_{N1}, 10_{N}. In some embodiments, the cycling through the X-ray sources 10 includes performing one cycle through all the X-ray sources 10 in a time interval of 0.2 seconds or less.

In the tomosynthesis X-ray imaging data acquisition process 104 just described, typically the active X-ray source will be a single one X-ray source. However, if the individual X-ray sources produce unsuitably low X-ray intensity to be used individually in the cycling, then the active X-ray source could be a subset of the X-ray sources 10 that are physically grouped close enough together to have a common effective projection view. The X-ray imaging data is then acquired along the projection view corresponding to the active X-ray source 10 that is switched on.

At an operation 104, the acquired X-ray imaging data is grouped into acquisition time intervals. Each acquisition time interval contains in some embodiments X-ray images from a contiguous set of N projections. To do the grouping, a synchronization module 22 is configured to bin the acquired X-ray imaging data into time bins or by interpolating the acquired X-ray imaging data to discrete times, or to a common time within the time interval. The groups need not be mutually exclusive (e.g., data near a boundary between two time intervals might be assigned to both time intervals). The output of this grouping operation 104 is an X-ray imaging data readout 24. As shown in FIG. 1, in the illustrative embodiment the electronic X-ray source switch 20 and the synchronization module 22 are disposed in the gantry 14 of the medical imaging system 1.

At an operation 106, the electronic controller 16 is configured to perform tomosynthesis image reconstruction of the X-ray imaging data readout 24 to generate at least one volumetric image. In some embodiments, tomosynthesis image reconstruction 106 of the X-ray imaging data grouped into each time interval is performed to generate a time sequence of volumetric images. In some examples, the tomosynthesis image reconstruction 106 of the data in a given time interval can include filtered back-projection, an iterative expectation-maximization (EM) algorithm, or another inverse Radon transform.

At an operation 108, the resulting volumetric images are spatially registered (i.e., aligned) to produce a time sequence of volumetric images that are spatially aligned. The spatial registration can be, for example, performing an elastic spatial image registration of the volumetric images.

Typically, the lung perfusion and/or ventilation images are desired to be constructed at one or more specific slices or slabs, e.g., at one slice or slab passing through the left lung L_{L} and another slice or slab passing through the right lung L_{R}. Hence, at an operation 110, a time sequence of spatially aligned images is produced by extracting at least one slice or slab passing through the at least one in vivo lung L from the volumetric images of the time sequence of spatially aligned volumetric images. The spatially aligned images of the time sequence of spatially aligned images are the extracted slice or slab images depicting a portion of the at least one in vivo lung. In some examples, the selected slice or slab images can be 2D slice images, or 3D volume images (i.e., chosen to exclude ribs/spine of the patient P). In another approach, subsequent image processing can be performed to virtually remove the ribs/spine.

The result of the foregoing processing is a time sequence of spatially aligned images 112 that depict at least one in vivo lung L of the patient P.

Continuing now with the process flow depicted by the flowchart of FIG. 3, at an operation 114, the electronic processor 16 is configured to identify temporal changes in the time sequence of spatially aligned images caused by respiration. For use, one or more sensors can include, for example, an electrocardiography (ECG) sensor 28 to generate an image sequence containing principally ventilation information. The ECG sensor 28 can be electronically connected to the electronic processor via one or more electronic components (not shown in FIG. 1 for clarity). In some embodiments, this is achieved by applying a low pass filter to the images with a filter cutoff frequency f_{c} that is less than the heart rate (HR) data acquired by an electrocardiography (ECG) sensor 28 to generate the image sequence containing principally ventilation information, for use in generating the ventilation image. Note that while in the processing sequence shown in FIGS. 2 and 3, the filtering 114 is performed after the slice or slab extraction operation 110, in a variant embodiment the filtering 114 could be performed on the volumetric images produced by the operation 108 after which the slice or slab extraction operation 110 would be applied to the filtered images.

At an operation 116, the electronic processor 16 is configured to identify temporal changes in the time sequence of spatially aligned images caused by perfusion. In some embodiments, this is achieved by applying a high pass filter to the images with a filter cutoff frequency f_{c} that is greater than the HR data acquired by the ECG sensor 28 to generate an image sequence containing principally perfusion information. It will be appreciated that the operations 114 and 116 can be performed concurrently or consecutively in either order. Again note that while in the processing sequence shown in FIGS. 2 and 3, the filtering 116 is performed after the slice or slab extraction operation 110, in a variant embodiment the filtering 116 could be performed on the volumetric images produced by the operation 108 after which the slice or slab extraction operation 110 would be applied to the filtered images.

At an operation 118, a ventilation image is generated based on voxel intensity variation over time of the time sequence of spatially aligned images after temporal filtering to suppress voxel intensity variation over time due to perfusion of the at least one in vivo lung L. For use in generating the ventilation image, one or more sensors can include, for example, a respiration rate (RR) monitor 30 attached to the patient via a belt 32. The RR monitor 30 can be electronically connected to the electronic processor via one or more electronic components (not shown in FIG. 1 for clarity). For mechanically ventilated patients, the RR data are provided in some embodiments by the mechanical ventilator 2. In some examples, respiration rate (RR) data from an RR monitor 30 connected to the patient P can be used to determine a respiratory cycle of the patient. To this end, as shown in FIG. 1, one or more sensors are attached to the patient P while the X-ray imaging data is acquired.

At an operation 120, a perfusion image is generated based on voxel intensity variation over time of the time sequence of spatially aligned images after temporal filtering to suppress voxel intensity variation over time due to ventilation of the at least one in vivo lung L. In some examples, the HR data from the ECG sensor 28 can be used to determine a cardiac cycle of the patient. To this end, as shown in FIG. 1, the one or more sensors are attached to the patient P while the X-ray imaging data is acquired can include, for example, an electrocardiogram (ECG) sensor 28 configured to acquire heart rate (HR) data of the patient P. The ECG sensor 28 can be electronically connected to the electronic processor via one or more electronic components (not shown in FIG. 1 for clarity).

At an operation 122, the ventilation image (from the operation 118) and the perfusion image (from the operation 120) can be displayed on the display device 18, e.g., side-by-side to enable a clinician to easily compare the ventilation and perfusion images. In a variant embodiment, the ventilation image and the perfusion image may be fused to generate a single fused image depicting both the ventilation and perfusion information. For example, the fused image could display ventilation information extracted from the ventilation image in a first color (e.g., green) and the perfusion information extracted from the perfusion image in a second color (e.g., red). The fused image is suitably displayed on the display device 18.

While the processing of FIG. 3 depicts processing to generate both a ventilation image (operations 114 and 118) and a perfusion image (operations 116 and 120), if the clinical objective is satisfied by only one of these images, then the other can be omitted. For example, if only a ventilation image is desired then the perfusion image operations 116 and 120 can be omitted; conversely, if only a perfusion image is desired then the ventilation image operations 114 and 118 can be omitted.

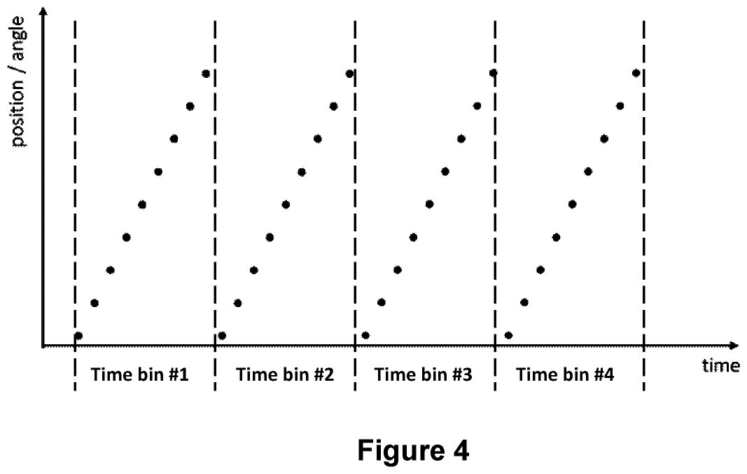

FIG. 4 shows an example representation for an X-ray imaging data acquisition scheme performed in the operation 10₂. In order to implement dynamic tomosynthesis, the x-ray sources need to be switched on periodically. As shown in FIG. 4, over time (i.e., x-axis) the X-ray sources 10₁, 10₂, . . . , 10_{N-1}, 10_{N}, which are located at different positions (linear tomosynthesis) or angles (circular tomosynthesis) are periodically activated (i.e., y-axis). FIG. 4 also diagrammatically shows one implementation of the grouping operation 104 by indicating suitable acquisition time intervals (referred to in FIG. 4 as “Time bin #1”, Time bin #2″ . . . ) into which the acquired X-ray data may be grouped. In this example, each cycle through the N X-ray sources corresponds to a single acquisition time interval for the grouping. This provides the highest temporal resolution.

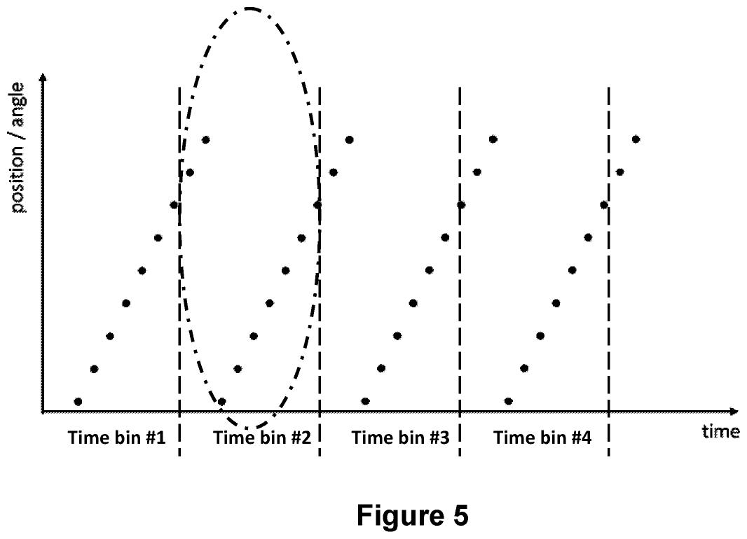

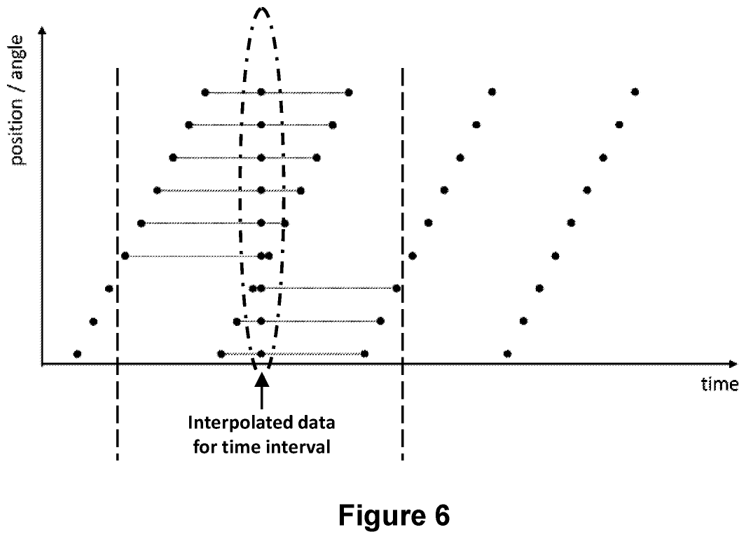

This acquisition scheme allows for a so-called sliding window image reconstruction. Two embodiments for this aspect are shown in FIGS. 5 and 6. The grouping approach illustrated in FIG. 5 provides the best temporal resolution, whereas the grouping approach illustrated in FIG. 6 provides more consistent projections which will lead to less image artifacts due to residual motion during the acquisition period.

As shown in FIG. 5, the system 1 has nine X-ray sources 10 (that is, N=9), which are activated over time as illustrated in FIG. 4. Any subsequent nine x-ray images (e.g., the dots enclosed in the dashed circle) can be used for the tomosynthesis reconstruction operation 106.

As shown in FIG. 6, the system 1 has nine X-ray sources 10, which are activated over time as illustrated in FIG. 4. Projections at a certain desired time are interpolated from the acquired data, e.g., using linear interpolations between the two nearest neighbors from the same angular/spatial position, to form interpolated data for the time interval as indicated in FIG. 6.

Reconstructed images from a tomosynthesis acquisition have anisotropic spatial resolution, in that the resolution perpendicular to the optical axis (that is, perpendicular to the general direction extending between the X-ray sources 10 and X-ray detector array 12, i.e., the left-right axis in the example shown in FIG. 1) of the system is given by the detector resolution; while the resolution parallel to the optical axis (that is, along the general direction extending from the X-ray sources 10 and X-ray detector array 12) is limited by the so-called tomo-angle, i.e., the angular range over which an image point has been seen during the acquisition. Therefore, the reconstructed image output by the operation 106 is often said to be “2.5D.” Once a time-series of 2.5D volumes has been reconstructed, processing in accord with perfusion and ventilation imaging in dynamic x-ray imaging is applied, e.g., as per the operations subsequent to operation 106 in FIGS. 2 and 3.

Elastic image registration 108 is applied to compensate for the organ motion. Note that this step is much more accurate than in dynamic x-ray imaging that does not employ tomosynthesis, since 2.5D images are available and provide additional information.

Analysis of the temporal change of the attenuation within each “voxel” of the registered time series of 2.5D volumes is performed. High-frequency changes (roughly above the heart frequency of 1 Hz) are related to perfusion. Low-frequency changes are related to ventilation. Hence, the filtering operation 114 (for ventilation imaging) or the filtering operation 116 (for perfusion imaging) is applied, and the ventilation image (operation 118) or perfusion image (operation 120) is obtained based on the temporal change of the attenuation within each “voxel” of the registered and filtered time series of 2.5D volumes.

The outputs of the filtering operations 114, 116 can be the ventilation image (operation 118) or perfusion image (operation 120), or corresponding perfusion and ventilation maps by dynamic X-ray imaging (i.e., both 2D and tomosynthesis X-ray imaging).

The illustrative example of FIG. 3 generates the ventilation image by low-pass filtering 114 and analyzing the resulting voxel intensity variation over time (and analogously for the perfusion image per operations 116 and 118). Alternatively, to the illustrative filtering approach, other approaches can be used to separate changes due to respiration and due to perfusion in the time series of 2.5D volumes:

For example, in another illustrative approach, for a time series of previously-registered images, pixel values in the time series of images are denoted as Mᵢₜ, where i is a spatial index, running over all pixels of the (possibly spatially down-sampled) detector and t is a temporal index. Instead of the separating perfusion and ventilation images via temporal low- and high-pass filtering, a maximum-likelihood approach can be implemented to estimate the desired information, where the a priori knowledge regarding the temporal behavior is coded in a regularization term. Without loss of generality, the to-be-estimated contribution of the blood to the measurements can be denoted as Pᵢₜ and the contribution of the ventilation can be denoted as Vᵢₜ. These are temporally varying values on top of a static background Bᵢ.

The maximum likelihood estimate is usually formulated as an optimization problem, containing a data term (i.e., the unknowns shall fit the measurements) and a regularization term (i.e., the data shall fit a priori knowledge). The data term is usually a least squares type term as shown in Equation (1):

$${\sum }_{i , t} {( {M}_{it} - {B}_{i} - {P}_{it} - {V}_{it} )}^{2} \qquad ( 1 )$$

The regularization term often codes temporal smoothness, as shown in Equation (2):

$${R}_{T} ( P ) = {\sum }_{t} {( {P}_{it} - {P}_{i , t + 1} )}^{2} \qquad ( 2 )$$

The regularization term often codes spatial smoothness, as shown in Equation (3):

$${R}_{S} ( P ) = {\sum }_{i} {\sum }_{j ∈ {N}_{i}} {( {P}_{it} - {P}_{jt} )}^{2} \qquad ( 3 )$$

where Nᵢ is the set of neighbors of pixel i. In Equations 2 and 3, the sum of squares is used, but other functions to penalize differences between spatially or temporally neighboring pixels can also be used (i.e., the absolute difference or the Huber function).

In some embodiments, the periodicity with the heart- and/or breathing cycle can also be used. This can be achieved with additional regularization terms of the form as shown in Equation (4):

$${R}_{C} ( P , T ) = {\sum }_{t} {( {P}_{it} - {P}_{i , t + T} )}^{2} \qquad ( 4 )$$

where T is the cycle time. The estimate for P and V is finally obtained by minimizing the cost function as shown in Equation (5):

$${Δ}^{2} ( B , P , V ) = {\sum }_{i , t} {( {M}_{it} - {B}_{i} - {P}_{it} - {V}_{it} )}^{2} + {α}_{1} {R}_{C} ( V , {T}_{P} ) + {α}_{2} {R}_{C} ( V , {T}_{V} ) + {α}_{3} {R}_{S} ( P ) + {α}_{4} {R}_{S} ( V ) + {α}_{5} {R}_{T} ( P ) + {α}_{6} {R}_{T} ( V ) \qquad ( 5 )$$

where αₖ denotes regularization parameters which influence how strong the a priori knowledge is. For instance, the temporal smoothness of the ventilation signal is known to be much stronger than the temporal smoothness of the perfusion image. Thus, the natural choice will be α₅«α₆.

In order to simplify the optimization problem, several simplifications are possible. In one example, the approach may operate on single pixels independently and smoothness can be obtained spatial low-pass filtering of either the input data M or the estimated ventilation and perfusion values. In another example, the approach may be split into two optimization problems by performing like in the original approach first a low- and high pass filtering of the measured data. For instance, if \hat{M}ᵢₜ denotes the low-pass filtered data, then the ventilation values Vᵢₜ may be estimated via minimizing as shown in Equation (6):

$${\sum }_{i , t} {( {\hat{M}}_{it} - {B}_{i} - {V}_{it} )}^{2} + {α}_{2} {R}_{C} ( V , {T}_{V} ) + {α}_{4} {R}_{S} ( V ) + {α}_{6} {R}_{T} ( V ) \qquad ( 6 )$$

and correspondingly the perfusion may be estimated from the high-pass filtered measurements \hat{M}ᵢₜ by minimizing as shown in Equation (7):

$${\sum }_{i , t} {( {\tilde{M}}_{it} - {P}_{it} )}^{2} + {α}_{1} {R}_{C} ( P , {T}_{P} ) + {α}_{3} {R}_{S} ( P ) + {α}_{5} {R}_{T} ( P ) \qquad ( 7 )$$

The final step of the filtering operations 114, 116 in this embodiment is to condense the temporally resolved estimate Pᵢₜ and Vᵢₜ into perfusion and ventilation maps. This can be done by first averaging the data over the respective cycles. For the perfusion case with a cycle period T_{P}, this is done as according to Equation (8):

$${\overline{P}}_{i} = \frac{1}{N} {\sum }_{j = 0}^{N - 1} {P}_{i , t + {jT}_{P}} \qquad ( 8 )$$

Next, a temporal gradient is calculated using Equation (9):

$${\overline{P}}_{i}^{′} = {\overline{P}}_{i + 1} - {\overline{P}}_{i} \qquad ( 9 )$$

The ventilation map is calculate correspondingly using the respiratory cycle period Ty.

In one embodiment (not illustrated), the X-ray detector array 12 is placed below the patient P and the X-ray detectors 10 are located overhead, i.e., above the patient P. In this configuration, proper positioning of the X-ray detector array 12 and the X-ray source array 10 can be difficult. Alternatively, as shown in FIG. 1 the dynamic tomosynthesis approach can be implemented in a lateral geometry. In this embodiment, there is no need to manually place the X-ray detector array 12 below the patient P (rather, it is located to the side, as shown in FIG. 1). Advantageously, the entire source-detector arrangement can be mounted on a fixed arm (e.g., the illustrative overhead gantry 14 shown in FIG. 1) and be placed across the patient P and a bed on which the patient P lies.

In the illustrative lateral tomosynthesis imaging system 1 of FIG. 1, the viewing axis of the 2.5D volume will be in the sagittal direction as it should match the direction of low resolution. In this case, a single image will not provide insights about left-right asymmetry, which is often preferred by radiologists. In order to mitigate this short-coming, in some embodiments a dedicated viewing mode is implemented in which two sagittal planes are shown, which have the same distance to the central sagittal plane of the patient. For example, in the operation 110 one sagittal plane can pass through the left lung L_{L} and the other sagittal plane can pass through the right lung L_{R}. This viewing mode can also be applied to the derived images, namely the ventilation and perfusion images.

In some embodiments, while the use of small tubes with CNT emitters are preferred, it is alternatively possible to use the tube concept developed for electron beam CT (see, e.g., S. Kulkarni et al., “Electron Beam CT: A Historical Review” American Journal of Roentgenology. 2021; 216:1222-1228. 10.2214/AJR.19.22681), where a single cathode is used and the electron beam is electromagnetically moved along a large anode (in the CT case, it was a ring with approximately a meter in diameter). In this embodiment, each Xray source 10₁, 10₂ . . . , 10_{N1}, 10_{N} is one position of the electron beam on the large anode.

In some embodiments, the virtual trajectory (i.e., the path on which the small tubes are placed) is not limited to a line or a circle. Any trajectory (e.g., a circular arc, a part of a helix) can be used. The sources 10 may also be distributed on a 2D surface.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A medical imaging system, comprising:
X-ray sources arranged to emit X-rays into an examination region along different respective projection views spanning less than 180 degrees;
an X-ray detector array arranged to detect the X-rays emitted by the X-ray sources after passing through the examination region; and
an electronic controller configured to perform an imaging method including:
acquiring X-ray imaging data by cycling through the X-ray sources with each step of the cycle including: switching an active X-ray source on to emit X-rays and the other X-ray sources off to not emit X-rays, and acquiring X-ray imaging data along the projection view corresponding to the active X-ray source that is switched on; and
performing tomosynthesis image reconstruction of the X-ray imaging data to generate at least one image.

2. The medical imaging system of claim 1, wherein the cycling through the X-ray sources includes performing one cycle through all the X-ray sources in a time interval of 0.2 seconds or less.

3. The medical imaging system of claim 1, wherein the tomosynthesis image reconstruction includes:
grouping the X-ray imaging data into acquisition time intervals; and
performing tomosynthesis image reconstruction of the X-ray imaging data grouped into each time interval to generate a time sequence of images.

4. The medical imaging system of claim 3, wherein the grouping includes interpolating the X-ray imaging data of each group to a common time within the time interval.

5. The medical imaging system of claim 3, wherein the generation of the time sequence of images includes:
spatially aligning the images of the time sequence of images.

6. The medical imaging system of claim 3, wherein the images of the time sequence of images are slice images, slab images, or volumetric images.

7. The medical imaging system of claim 3, wherein the time sequence of images depict at least one in vivo lung, and the imaging method further includes:
generating a perfusion image based on voxel intensity variation over time of the time sequence of images.

8. The medical imaging system of claim 3, wherein the time sequence of images depict at least one in vivo lung, and the imaging method further includes:
generating a ventilation image based on voxel intensity variation over time of the time sequence of images.

9. The medical imaging system of claim 1, wherein the X-ray sources are configured to be electronically switched on to emit X-rays and electronically switched off to not emit X-rays, and the X-ray sources have a switching speed of 100 milliseconds or faster.

10. The medical imaging system of claim 9, wherein the X-ray sources comprise cold-cathode X-ray tubes or carbon nanotube field emitter cold-cathode X-ray tubes.

11. The medical imaging system of claim 9, wherein the X-ray detector array comprises complementary metal oxide semiconductor (CMOS)-based detectors or direct conversion X-ray detectors.

12. The medical imaging system of claim 1, wherein the X-ray sources are arranged as a linear array or as a loop.

13. The medical imaging system of claim 1, further comprising:
an overhead gantry having first and second descending arms spaced apart to accommodate an associated human subject disposed in a prone or supine position between the first and second descending arms;
wherein the X-ray sources are disposed on the first descending arm

14. A medical imaging method performed using X-ray sources arranged to emit X-rays into an examination region along different respective projection views spanning less than 180 degrees, and an X-ray detector array arranged to detect the X-rays emitted by the X-ray sources after passing through the examination region, the medical imaging method comprising:
cycling through the X-ray sources with each step of the cycle including:
switching an active X-ray source on to emit X-rays and the other X-ray sources off to not emit X-rays, and
acquiring X-ray imaging data along the projection view corresponding to the active X-ray source that is switched on; and
performing tomosynthesis image reconstruction of the acquired X-ray imaging data to generate at least one image.

15. The medical imaging method of claim 14, wherein:
the cycling through the X-ray sources includes performing one cycle through all the X-ray sources in a time interval of 0.2 seconds or less;
the tomosynthesis image reconstruction includes grouping the X-ray imaging data into acquisition time intervals and performing tomosynthesis image reconstruction of the X-ray imaging data grouped into each time interval to generate a time sequence of images; and
the medical imaging method further includes:
producing a time sequence of spatially aligned images from the time sequence of images by operations including at least performing elastic spatial image registration of the images;
generating a perfusion image based on voxel intensity variation over time of the time sequence of spatially aligned images; and
generating a ventilation image based on voxel intensity variation over time of the time sequence of spatially aligned images.
